(19) United States
(12) Patent Application Publication
(10) Pub. No.: US 2023/0355549 A1
(43) Pub. Date: Nov. 9, 2023

(54) METHOD OF PREVENTING KIDNEY INJURY DISRUPTION OF INTESTINAL LYMPHATICS

(71) Applicant: Vanderbilt University, Nashville, TN (US)

(72) Inventors: Valentina Kon, Nashville, TN (US); Naji Abumrad, Nashville, TN (US); Sean Davies, Nashville, TN (US); Haichun Yang, Nashville, TN (US)

(21) Appl. No.: 18/134,539

(22) Filed: Apr. 13, 2023

Related U.S. Application Data
(63) Continuation of application No. PCT/US21/54872, filed on Oct. 13, 2021.
(60) Provisional application No. 63/091,052, filed on Oct. 13, 2020.

(51) Int. Cl. A61K 31/135; A61P 13/12

(52) U.S. Cl. CPC A61K 31/135; A61P 13/12

(57) ABSTRACT

A method of treating proteinuric kidney injury, comprising administering an effective isoLG scavenging amount of at least one compound of the present invention.

### PRIOR APPLICATIONS

This application is a continuation-in-part International Patent Application No. PCT/US2021/054872, filed Oct. 13, 2021, which claims benefit to U.S. Provisional Application No. 63/091,052 filed Oct. 13, 2020, the entire disclosures of which are incorporated herein by this reference.

### GOVERNMENT SUPPORT

This invention was made with government support under grant number NIH 1P01HL116263 awarded by the National Institutes of Health. The government has certain rights to this invention.

### BACKGROUND OF THE INVENTION

Kidney disease is known to affect the structure and function of the intestines. Although intestinal lymphatics are central in absorption and remodeling of dietary and synthesized lipids/lipoproteins, very little is known about whether and how kidney injury impacts the intestinal lymphatic network, or the lipoproteins transported therein. To examine the effects of kidney injury on intestinal lymphatic vessels and mesenteric lymph, the present inventors used two proteinuric models (puromycin aminoglycoside-treated rats and NEP25 transgenic mice).

The present inventors have discovered that kidney injury expanded the intestinal lymphatic network, activated the lymphatic endothelial cells, and increased the mesenteric lymph flow. The lymph of kidney-injured animals contained increased levels of cytokines, immune cells, and greater output of apolipoprotein AI (apoAI). Moreover, an intestinally originating uremic toxin, indoxyl sulfate, stimulated ileal organoid production of reactive dicarbonyls, e.g. isolevuglandin (IsoLG). IsoLG was increased in the ileum and in the mesenteric lymph. IsoLG-modified apoAI directly increased lymphatic vessel contractions, activated lymphatic endothelial cells, and induced lymphangiogenesis via VEGF-C secretion by macrophages. Thus, embodiments of the present invention include a novel mediator (IsoLG-modified apoAI) and a new pathway (intestinal lymphatic network) in the crosstalk between kidneys and intestines that underlies the adverse systemic consequences attending kidney disease.

Kidney disease is well-recognized as causing dysregulated levels, composition, and function of lipids and lipoproteins. Intestinal lymphatics are key in lipid absorption and transport/remodeling of lipoproteins. The present inventors have discovered that kidney injury stimulates intestinal lymphangiogenesis, activates lymphatic endothelial cells, increases mesenteric lymph flow and alters the composition of lymph including lipoproteins (HDL/apoAI) and inflammatory factors. Critically, kidney injury stimulates intestinal production of reactive dicarbonyls that modify HDL/apoAI, resulting in increased contractions of lymphatic vessels and activated lymphatic endothelial cells. These results provide a novel mediator and pathway in the kidney-gut crosstalk that underlies adverse consequences of kidney disease.

### SUMMARY OF THE INVENTION

The present invention meets a long felt need because chronic kidney disease (CKD) affected ˜9.1% of the global population, or 700 million people, in 2017. The prevalence of kidney disease and its related morbidity and mortality are increasing because of many factors, particularly the ageing population and growing prevalence of diabetes. The structure and function of many organs and tissues are disrupted in kidney disease, which causes complications, such as infection, cardiovascular disease (CVD), peripheral arterial disease, bone disease, anemia, and acute kidney injury, with associated increases in hospitalization and mortality.

The present invention also details the importance of kidney-gut cross-talk in CKD-related complications. Little consideration has previously been given to intestinal lymphatic changes in kidney injury or disease. This is despite that the intestinal lymphatics play a central role in immunity by providing a site for the dissemination and activation of immune/inflammatory cells and mediators. The intestinal lymphatics also transport dietary and endogenous lipids in the form of lipoproteins, including chylomicrons, very-low-density lipoproteins (VLDLs), and high-density lipoproteins (HDLs), which impact CVD progression. Furthermore, numerous recent studies have demonstrated that dysfunction of the lymphatics in specific organs potentiates a range of diseases, including cancers, CVD, autoimmune diseases, and neuro-degenerative diseases. The function and growth of renal lymphatics even appear to influence the progression of acute kidney injury and CKD, and out-comes following renal transplantation.

Using puromycin aminoglycoside (PAN)-treated rats, the present invention shows that following proteinuric kidney injury there are substantial increases in intestinal lymph flow rate (>5-fold), reductions in intestinal lymph albumin transport, and increases in intestinal lymph lipids and lipoproteins (particularly HDL and apolipoprotein AI [apoAI]) in parallel with similar changes in plasma. Also, data show that increases in T-helper 17 cells and cytokines, including interleukin-6, interleukin-10, and interleukin-17, in intestinal lymph but not plasma in PAN rats. In both PAN rats and Nphs1-hCD25 (NEP25) trans-genic mice (used as another model of proteinuric kidney injury), they show in-creases in mRNA expression of lymphatic endothelial cell (LEC) markers (including podoplanin, vascular endothelial growth factor receptor 3 [VEGFR3], and lymphatic endothelial receptor 1 [LYVE-1]) in the ileum, complemented by increases in podoplanin-positive lymphatic vessels in the ileum of PAN rats. Kidney injury in PAN rats also altered ileal LEC expression of key genes involved in vasodilation (e.g., increased endothelial-specific nitric oxide, Nos3) and immune cell chemoattraction (e.g., increased CCL21 and higher SPHK2 together with decreased SPNS2 expression, key regulators of sphingosine-1-phosphate [SP] production, which was increased in mesenteric lymph).

The present inventors also investigated how proteinuric kidney injury modifies intestinal lymph lipoproteins, and whether these changes regulate intestinal LEC and lymphatic vessels. Kidney injury in-creases oxidative stress and lipid peroxidation, leading to the generation of a range of lipid aldehydes, such as isolevuglandin (IsoLG). IsoLG is a highly reactive dicarbonyl that compromises apoAI function. Both PAN rats and NEP25 transgenic mice had elevated total IsoLGlysine in the ileum, and PAN rats had elevated IsoLG-lysine in the mesenteric lymph, but not plasma. IsoLG can be generated by the peroxidase enzyme myeloperoxidase (MPO), and this enzyme was elevated in the intestinal wall of the proteinuric rats. The reason why MPO activity was elevated in the intestine of proteinuric rats was not completely defined, and this could be the subject of future study. IsoLG was colocalized with apoAI in the ileum and intestinal lymph of PAN rats. IsoLG-modified apoAI, but not native apoAI, was shown ex vivo to directly increase lymphatic vessel contractions, activate LEC, and increase secretion of prolymphangiogenic factor vascular endothelial growth factor C (VEGF-C) from isolated macrophages. Treatment of the NEP25 mice with a dicarbonyl scavenger of the present invention reduced IsoLG in the ileum and reduced the lymphatic marker podoplanin in the intestine, supporting that IsoLG promoted the intestinal lymphatic vessel changes observed in the rodents with proteinuric kidney injury.

One aspect of the present invention is that intestinal lymphatic composition, structure, and function are extensively modified in rodent models of proteinuric kidney injury without renal failure. These changes play a role in modulating crosstalk between the kidneys, intestine, and other organs, which contributes to systemic complications in kidney disease.

Changes to lymphatic structure and function throughout the body have been shown to be important contributors to the progression and complications of kidney injury and disease. The present inventors identify that increased IsoLG-modified apoAI modulates intestinal lymphatic structure and contractions in rodents with kidney injury and that an inhibitor of IsoLG formation is able to reduce lymphangio-genesis. Thus, one aspect of the invention is this inhibitor impacting the progression and complications of kidney injury and disease. The present inventors also demonstrate that intestinal lymphatic lipid and lipoprotein trafficking are considerably modified with kidney injury. Systemic dyslipidemia is a significant risk factor for CVD; and normalization of intestinal lymph lipoprotein trafficking, is shown to be a treatment to reduce cardiovascular complications in patients with kidney injury and disease.

A major focus of experimental and clinical studies has been to understand the pathophysiology of interorgan crosstalk, especially between injured kidneys and the gut. Kidney disease is a strong modulator of the composition and metabolism of the intestinal microbiome that produces toxins such as phenols (p-cresyl sulfate), indoles (indoxyl sulfate) and trimethylamine N-oxide. Kidney injury also disrupts the intestinal barrier that promotes translocation of bacterial components and endotoxins into the circulation, which then initiate immune activation and proinflammatory signaling. The primary pathways for mediators in the kidney-gut crosstalk are thought to involve blood vessels and nerves. Little attention has been given to lymphatics. Intestinal lymphatics are unique in that in addition to clearing interstitial fluid, macromolecules, immune/inflammatory cells, they are responsible for absorption of dietary lipids and transport/remodeling of lipoproteins. Lymphatics are also a primary conduit for transport of high density lipoprotein (HDL) from the peripheral interstitium to the circulation. Disruptions in lymph transport and lymphatic vessel integrity have recently been recognized as powerful potentiators of disease, including cardiovascular disease (CVD), inflammatory bowel disease, and chronic kidney disease (CKD). Although the intestinal lymphatic vessel number and function are affected by inflammation and dyslipidemia, whether kidney injury, characterized by inflammation and abnormal lipid metabolism, affects intestinal lymphatics is unknown.

Abnormalities in levels and composition of plasma lipids/lipoproteins observed in many diseases have been ascribed to changes in production and modifications by the liver. By contrast, although intestines are also a key source of apoAI/HDL, there is little information about the intestinal contribution to abnormal lipoproteins prevailing in disease. Interestingly, intestinal microbial variation has recently been shown to modulate plasma levels of total cholesterol and LDL, and be especially important in metabolism of VLDL and HDL, including the reverse cholesterol transport function of HDL. This may be pathophysiologically germane since modifications in the structure and composition of apoAI/HDL determines the particle's beneficial/detrimental effects.

A key mechanism in lipoprotein modification involves adduction by reactive carbonyls including malondialdehyde, 4-hydroxynonenal, 4-oxo-neonenal, and the most reactive among all the carbonyls, isolevuglandin (IsoLG). While individual carbonyls can affect specific apoAI/HDL functions, IsoLG impairs the fundamental actions of apoAI: cholesterol efflux, anti-inflammation, and anti-oxidation. IsoLG is also effective at 10-30 times lower levels than other carbonyls in changing apoAI functions. Kidney disease alters the composition and functionality of HDL and increases plasma protein adducts. The present inventors have shown that HDL particles become dysfunctional by apoAI modification with IsoLG, which impairs apoAI/HDL capacity to facilitate cholesterol efflux from macrophages and not only reduces HDL's ability to inhibit cytokine induction but potentiates LPS-induced IL-1β expression. One of ordinary skill in the art can expect that intestines are not only a source of apoAI/HDL but also a site of apoAI/HDL modifications that can have pathophysiologic implications, including regulation of lymphangiogenesis.

The present inventors show that dyslipidemia and inflammation prevailing in kidney disease impact the structure and function of intestinal lymphatics and embodiments of the present invention are treating, preventing and/or ameliorating this effect.

Thus, disclosed is a method for treating preventing and/or ameliorating the effect of kidney disease on the structure and function of intestinal lymphatics comprising identifying a subject with kidney disease, and administering to said subject an effective isoLG scavenging amount of at least one compound of the following formula:

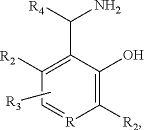

wherein R is N or C—R₂; R₂ is independently selected from H, substituted or unsubstituted C₁-C₁₀ alkyl, C₁-C₁₀ alkoxy, hydroxymethyl, hydroxy; R₃ is H, halogen, C₁-C₁₀ alkyl, alkoxy, hydroxyl, nitro; R₄ is H, substituted or unsubstituted C₁-C₁₀ alkyl, carboxyl; and pharmaceutically acceptable salts thereof.

Also disclosed is a method of modulating intestinal lymphatic function to ameliorate kidney injury or disease, comprising administering an effective isoLG scavenging amount of at least one compound of the following formula:

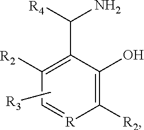

wherein R is N or C—R₂; R₂ is independently selected from H, substituted or unsubstituted C₁-C₁₀ alkyl, C₁-C₁₀ alkoxy, hydroxymethyl, hydroxy; R₃ is H, halogen, C₁-C₁₀ alkyl, alkoxy, hydroxyl, nitro; R₄ is H, substituted or unsubstituted C₁-C₁₀ alkyl, carboxyl; and pharmaceutically acceptable salts thereof

Also disclosed is a method of ameliorating systemic complications of kidney injury or disease, comprising administering an effective isoLG scavenging amount of at least one compound of the following formula:

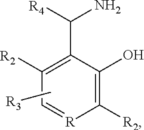

wherein R is N or C—R₂; R₂ is independently selected from H, substituted or unsubstituted C₁-C₁₀ alkyl, C₁-C₁₀ alkoxy, hydroxymethyl, hydroxy; R₃ is H, halogen, C₁-C₁₀ alkyl, alkoxy, hydroxyl, nitro; R₄ is H, substituted or unsubstituted C₁-C₁₀ alkyl, carboxyl; and pharmaceutically acceptable salts thereof

Also disclosed is a method of ameliorating intestinal lymphatic dysfunction, comprising administering an effective isoLG scavenging amount of at least one compound of the following formula:

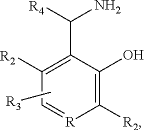

wherein R is N or C—R₂; R₂ is independently selected from H, substituted or unsubstituted C₁-C₁₀ alkyl, C₁-C₁₀ alkoxy, hydroxymethyl, hydroxy; R₃ is H, halogen, C₁-C₁₀ alkyl, alkoxy, hydroxyl, nitro; R₄ is H, substituted or unsubstituted C₁-C₁₀ alkyl, carboxyl; and pharmaceutically acceptable salts thereof

In the methods of the present invention, the compound may also be of the following formula:

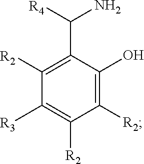

wherein R₂ is independently chosen from H, substituted or unsubstituted alkyl; R₃ is H, halogen, alkyl, alkoxy, hydroxyl, nitro; R₄ is H, substituted or unsubstituted alkyl, carboxyl; and pharmaceutically acceptable salts thereof.

In one embodiment, R₂ is independently chosen from H, ethyl, methyl.

In another embodiment, the compound is 2-hydroyxbenzylamine, methyl-2-hydroyxbenzylamine, ethyl-2-hydroyxbenzylamine.

In another embodiment, the compound is:

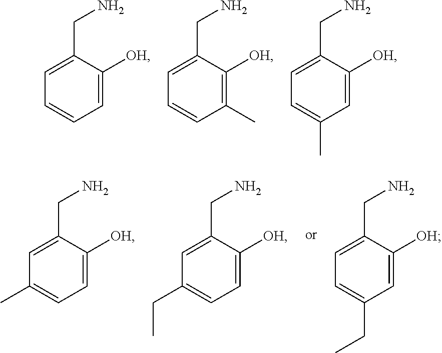

or a pharmaceutically acceptable salt thereof.

In another embodiment, the compound is:

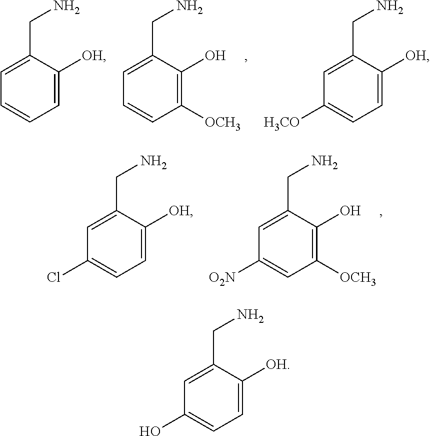

or a pharmaceutically acceptable salt thereof.

In another embodiment, the compound or pharmaceutically acceptable salt thereof is administered in a composition that comprises said compound or a pharmaceutically acceptable salt thereof and a pharmaceutically acceptable carrier.

In other embodiment, the compound or pharmaceutically acceptable salt thereof is administered is co-administered with another active agent that had a known side effect of treating damage from kidney disease and/or inflammation.

Additional advantages and embodiments of the present invention will be set forth in the description which follows, and in part will be obvious from the description, or can be learned by practice of the invention. The advantages and embodiments of the present invention will be realized and attained by means of the elements and combinations particularly pointed in the appended claims. It is to be understood that both the foregoing general description and the following more detailed description are exemplary and exemplary only and are not restrictive of the invention, as claimed.

### BRIEF DESCRIPTION OF THE DRAWINGS

FIG. 1 shows that proteinuric kidney injury increases mesenteric lymph flow and changes lymph composition. (A) Lymphatic flow rate in mesenteric vessels was consistently higher in PAN-injured animals vs controls. (B) Albumin concentration and output in mesenteric lymph were significantly decreased in PAN vs controls. (C) Cholesterol and triglyceride concentrations in mesenteric lymph were lower in PAN; total output of cholesterol and triglycerides in mesenteric lymph was significantly higher in PAN vs controls. (D) NMR analysis of lipoprotein particles in mesenteric lymph showed similar LDL particles, smaller triglyceride (TRL)-containing particles and larger HDL particles in PAN vs controls. (E) Size-exclusion chromatography (SEC) on the FPLC systems found PAN increased protein, cholesterol, and phospholipids in fractions coinciding with spherical HDL and chylomicrons and increased triglycerides in fractions corresponding to chylomicrons. (F) Lymph apoAI concentration was similar in PAN vs controls; total mesenteric lymph apoAI output was increased in PAN vs controls. (G) Plasma apoAI concentration was increased in PAN vs controls. (H) Double staining of ileal tissue with apoAI (red) and podoplanin (green) showed PAN redistributed apoAI toward the lacteals.

FIG. 2 shows that proteinuric kidney injury alters immune cells and cytokines in mesenteric lymph. (A) Flow cytometry of mesenteric lymph showed more Th17 cells (CD3+/CD4+/CCR6+) in lymph of PAN vs controls. (B) Mesenteric lymph showed more IL-6, IL-10 and IL-17 in PAN vs controls, while IL-1 was not different.

FIG. 3 shows that proteinuric kidney injury expands the mesenteric lymphatic vascular network and activates lymphatic endothelial cells (LECs). (A) PAN increased ileal expression of lymphangiogenic factors, including podoplanin (PDPN), LYVE-1 (LYVE1) and VEGFR3 (FLT4) mRNA. (B) Staining of PAN injured rats showed increased podoplanin expression vs controls. (C) NEP25 increased ileal gene expression of podoplanin (PDPN) and VEGFR3 (FLT4). (D) Staining of NEP25 ileum showed increased podoplanin expression compared to control mice. (E) PAN increased eNOS (Nos3) mRNA expression in ileal podoplanin-positive LECs vs controls. (F) PAN kidney injury significantly increased expression of the chemoattractant CCL21 mRNA in ileal podoplanin-positive LECs. (G) Podoplanin-positive LECs isolated from ilea of PAN showed greater SPHK2 mRNA and less SPNS2 mRNA vs ilea of normal controls. (H) Mesenteric lymph from PAN rats had more SP than control lymph.

FIG. 4 shows that proteinuric kidney injury stimulates ileal production of IsoLG. (A) PAN increased IsoLG adducts in ileal tissue vs controls. (B) PAN mesenteric lymph contained more IsoLG adducts compared to controls. (C) Cultured enteroids exposed to the uremic toxin indoxyl sulfate (IS) produced more IsoLG adducts vs vehicle. (D) Double staining of apoAI (green) and IsoLG (red) in ileum of PAN showed IsoLG adducts in lacteals that colocalized with apoAI (arrows).

FIG. 5 shows that IsoLG modified apoAI activates cultured lymphatic endothelial cells (LECs) and alters vasodynamics of isolated mesenteric lymph vessels. In vitro, cultured LECs exposed IsoLG-apoAI produced (A) more ROS and (B) increased eNOS (Nos3) gene expression vs unmodified apoAI. IsoLG-apoAI (C) increased contraction frequency from baseline, (D) did not change the end systolic diameter, (E) reduced end diastolic diameter from baseline, and (F) reduced contraction amplitude from baseline vs unmodified apoAI.

FIG. 6 shows that proteinuric kidney injury stimulates ileal macrophage production of VEGF-C. (A) PAN increased ileal VEGF-C vs controls. (B) VEGF-C concentration in PAN lymph was lower but total output of VEGF-C was significantly greater in PAN vs controls. (C) Double staining of ileum with VEGF-C (red) and CD68 (green) showed greater number of CD68-positive cells co-localized with VEGF-C (arrows) in PAN vs controls. (D) Cultured macrophages exposed to IsoLG-apoAI expressed more VEGFC mRNA vs unmodified apoAI.

FIG. 7 shows treatment with a compound of the present invention decreased ileal lymphangiogenesis and IsoLG adducts. (A) PPM significantly reduced intestinal lymphangiogenesis in proteinuric NEP25 mice. (B) PPM also decreased IsoLG adduct in ileum of NEP25 mice.

FIG. 8 shows treatment with a compound of the present invention reduces IsoLG-lysine in mesenteric lymph.

### DESCRIPTION OF THE INVENTION

The present inventors have discovered compounds that are effective in the treatment of the effects of kidney damage

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “subject” refers to a target of administration. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. As can be seen herein, there is overlap in the definition of treating and preventing.

As used herein, the term “diagnosed” means having been subjected to a physical examination by a person of skill, for example, a physician, and found to have a condition that can be diagnosed or treated by the compounds, compositions, or methods disclosed herein. As used herein, the phrase “identified to be in need of treatment for a disorder,” or the like, refers to selection of a subject based upon need for treatment of the disorder. For example, a subject can be identified as having a need for treatment of a disorder (e.g., a disorder related to inflammation) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “scavenger” or “scavenging” refers to a chemical substance that can be administered in order to remove or inactivate impurities or unwanted reaction products. For example, without being bound by theory or mechanism, the IsoLGs irreversibly adduct specifically to lysine residues on proteins. The IsoLGs scavengers of the present invention react with IsoLGs before they adduct to the lysine residues. Accordingly, the compounds of the present invention “scavenge” IsoLGs, thereby preventing them from adducting to proteins.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by a formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. One example is —O-pentyl. “Alkoxy” also includes polymers of alkoxy groups as just described; that is, an alkoxy can be a polyether such as —OA¹—OA² or —OA¹—(OA²)ₐ—OA³, where “a” is an integer of from 1 to 200 and A¹, A², and A³ are alkyl and/or cycloalkyl groups.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “hydroxyl” as used herein is represented by a formula —OH.

The term “nitro” as used herein is represented by a formula —NO₂.

The term “pharmaceutically acceptable” describes a material that is not biologically or otherwise undesirable, i.e., without causing an unacceptable level of undesirable biological effects or interacting in a deleterious manner.

Examples of compounds of the present invention include, but are not limited to, compounds selected from the formula:

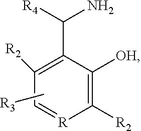

wherein: R is N or C—R₂; R₂ is independently H, substituted or unsubstituted C₁-C₁₀ alkyl, C₁-C₁₀ alkoxy, hydroxy; R₃ is H, halogen, C₁-C₁₀ alkyl, alkoxy, hydroxyl, nitro; R₄ is H, substituted or unsubstituted C₁-C₁₀ alkyl, carboxyl; and pharmaceutically acceptable salts thereof.

Further example include compounds of the following formula:

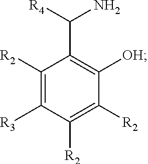

wherein:
R₂ is independently chosen from H, substituted or unsubstituted alkyl;
R₃ is H, halogen, alkyl, alkoxy, hydroxyl, nitro;
R₄ is H, substituted or unsubstituted alkyl, carboxyl; and pharmaceutically acceptable salts thereof.

In other embodiments, R₂ is independently chosen from H, ethyl, methyl.

In other embodiments, the compound may be chosen from:

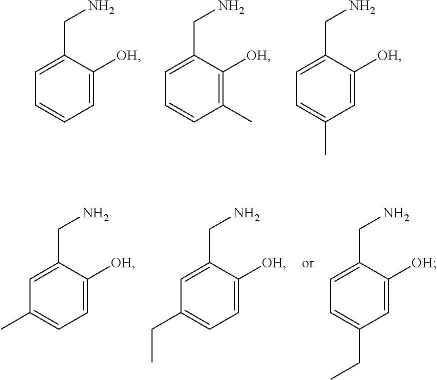

or a pharmaceutically acceptable salt thereof.

The compound may also be chosen from:

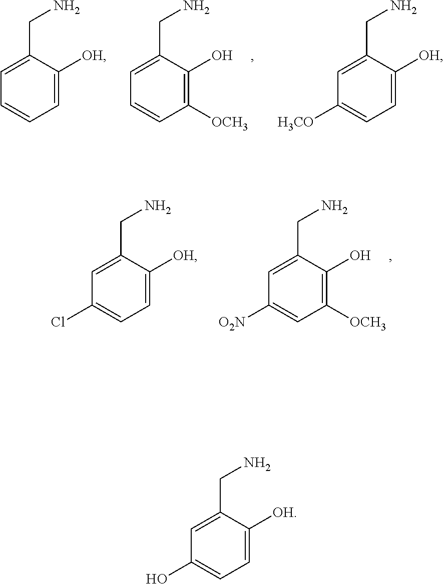

or a pharmaceutically acceptable salt thereof.

The compounds may also be chosen from:

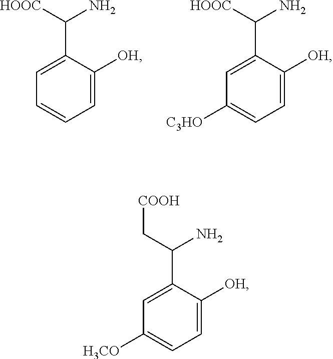

or a pharmaceutically acceptable salt thereof.

The compounds may also be chosen from

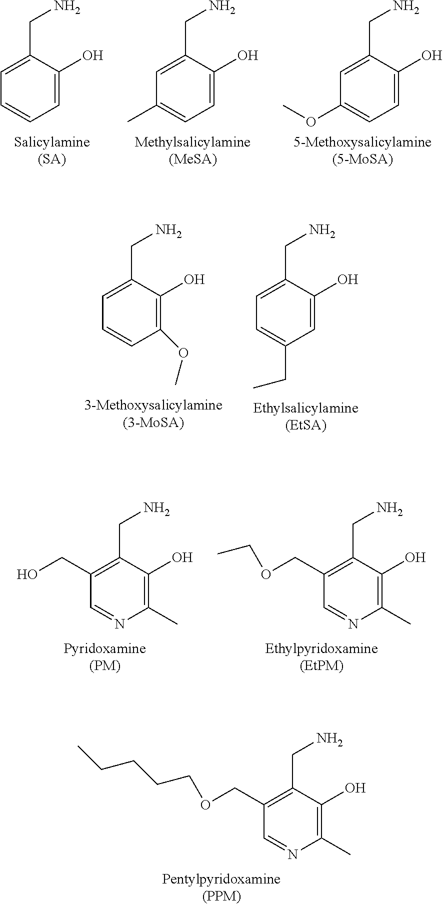

or a pharmaceutically acceptable salt thereof.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

Accordingly, one embodiment of the present invention is a method of treating proteinuric kidney injury, comprising administering to a patient in need thereof an effective amount of at least one IsoLG scavenger compound of the present invention, or a pharmaceutically acceptable salt thereof. Preferably, the compound is 2-HOBA, methyl-2-HOBA or ethyl-2-HOBA.

Another embodiment of the present invention is a method of treating damage from kidney disease. In aspects of the invention, the damage is to the intestinal lymphatic network. In other aspects, the damage is increased contractions of lymphatic vessels and activated lymphatic endothelial cells. In other aspects, the damage is disruptions in lymph transport and lymphatic vessel integrity.

Another embodiment of the present invention is a method of treating proteinuric kidney injury, comprising identifying a subject in need of treatment of kidney damage; and administering to said subject an effective isoLG scavenging amount of at least one compound of the present invention.

In one aspect, the proteinuric kidney injury is damage from kidney disease. In another aspect, the damage is to the intestinal lymphatic network. In another aspect, the damage is increased contractions of lymphatic vessels and activated lymphatic endothelial cells. In yet another aspect, the damage is disruptions in lymph transport and lymphatic vessel integrity.

Another embodiment of the present invention is a method of modulating intestinal lymphatic function to ameliorate kidney injury or disease, comprising administering an effective isoLG scavenging amount of at least one compound of the present invention.

Another embodiment of the present invention is a method of ameliorating systemic complications of kidney injury or disease, comprising administering an effective isoLG scavenging amount of at least one compound of the present invention. In one aspect, the IsoLG is in the intestinal lymphatic network. In another aspect, the systemic complication is cardiovascular, circulatory, or obesity-related.

Another embodiment of the present invention is a method of ameliorating intestinal lymphatic dysfunction, comprising administering an effective isoLG scavenging amount of at least one compound of the present invention. In one aspect, the dysfunction is intestinal lymphangiogenesis.

The above embodiments comprise administering to a patient in need thereof an effective amount of at least one IsoLG scavenger compound of the present invention, or a pharmaceutically acceptable salt thereof. Preferably, the compound is 2-HOBA, methyl-2-HOBA or ethyl-2-HOBA.

As stated above, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention can comprise a compound of the invention (or pharmaceutically acceptable salts thereof) as an active ingredient, a pharmaceutically acceptable carrier, and optionally one or more additional therapeutic agents or adjuvants.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

It is understood that the disclosed compositions can be prepared from the disclosed compounds. It is also understood that the disclosed compositions can be employed in the disclosed methods of using.

Accordingly, the pharmaceutical compositions of the present invention include those that contain one or more other active ingredients, in addition to a compound of the present invention.

The above combinations include combinations of a disclosed compound not only with one other active compound, but also with two or more other active compounds. Likewise, disclosed compounds may be used in combination with other drugs that are used in the prevention, treatment, control, amelioration, or reduction of risk of the diseases or conditions for which disclosed compounds are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient can be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000 and any amount in-between, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent may be coadministered, either in concomitant therapy or in a fixed combination.

In addition to its superior safety profile, compounds of the present invention are also desirable for its feasibility of use. While an option for administration, compounds of the present invention do not have to be injected or infused, as they are orally bioavailable. Further, compounds of the present invention have a long shelf life at room temperature (≥2 years). Compounds of the present invention also can be manufactured at a substantially lower cost compared to biologic treatments, which will further lower patient burden and ensure access.

In another embodiment of the present invention, the compounds of the present invention can be co-administered to a patient in need thereof with another active ingredient that has a known side effect of treating kidney damage and/or inflammation.

That is, a compound of the present invention can be administered alone or in combination with an effective amount of at least one additional active agent. “Combined” or “in combination” or “combination” should be understood as a functional co-administration, wherein some or all compounds may be administered separately, in different formulations, different modes of administration (for example subcutaneous, intravenous or oral) and different times of administration. The individual compounds of such combinations may be administered either sequentially in separate pharmaceutical compositions as well as simultaneously in combined pharmaceutical compositions.

The present inventors have shown that proteinuric kidney injury increases mesenteric lymph flow and changes lymph composition. The present inventors used the well-established puromycin aminoglycoside nephropathy (PAN) model in the rat. As expected, PAN rats developed ascites, proteinuria, hypoalbuminemia, increased plasma cholesterol and triglycerides compared with controls (Table 1, below).

TABLE 1
Albumin/creatinine concentration ratio (ACR),
plasma albumin, plasma total cholesterol and
triglyceride concentration in Cont and PAN rats

| | | | Statistical |
|---|---|---|---|
| | Cont | PAN | significance |
| ACR (mg/mg) | 0.037 ± 0.003 | 126.652 ± 13.283 | p < 0.001 |
| Plasma Albumin | 523.8 ± 23.9 | 350.7 ± 27.3 | p < 0.001 |
| (mg/dL) | | | |
| Plasma Cholesterol | 44.0 ± 3.5 | 236.6 ± 36.8 | p < 0.001 |
| (mg/dL) | | | |
| Plasma Triglyceride | 34.9 ± 2.8 | 132.1 ± 19.2 | p < 0.001 |
| (mg/dL) | | | |

Data expressed as means ± SEM
n = 12 in each group

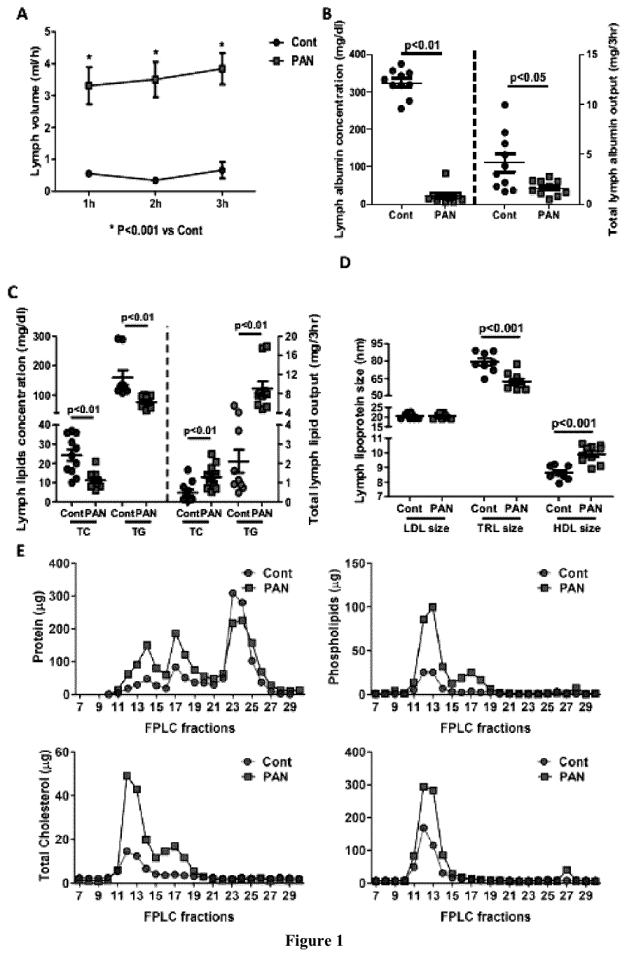

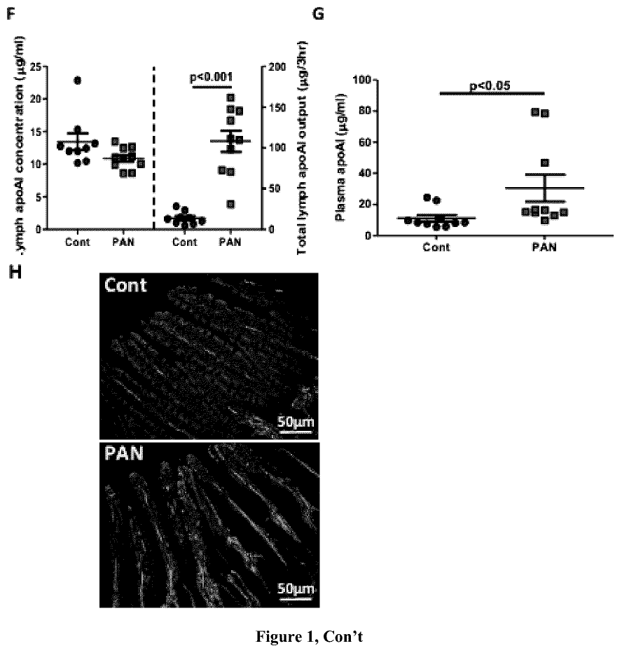

The proteinuric injury caused a striking increase in the mesenteric lymph flow (see FIG. 1A). Mesenteric lymph in PAN had reduced albumin, and despite increased lymph volume, total mesenteric albumin output was less in PAN vs controls (see FIG. 1B). Lymphatic concentrations of cholesterol and triglycerides were lower in PAN vs controls, however, total lymphatic output of these lipids increased, paralleling elevated plasma lipids (see FIG. 1C, Table 1). PAN did not affect lymphatic LDL particle size although triglyceride-containing particles corresponding to VLDL were smaller and HDL particles larger in lymph of PAN vs controls (see FIG. 1D). Further analysis of HDL particles showed increased total protein, total cholesterol, and phospholipids in fractions coinciding with spherical HDL (see FIG. 1E). The plasma HDL particle size was not changed (PAN: 10.7±0.1 vs Cont:10.5±0.1 nm, pNS).

Since 30% of apoAI in the circulation originates in the ileum, we also assessed the intestinal and lymphatic levels of apoAI. Ileal apoAI protein level were not different between the groups (PAN:1.06±0.06 vs Cont:1.16±0.17 μg/mg, pNS), although the total mesenteric output of apoAI was significantly higher in PAN vs controls (see FIG. 1F). This was attended by a higher level of plasma apoAI in PAN vs controls at this early stage of injury (see FIG. 1G). The ileum of PAN rats showed more prominent apoAI protein expression that was redistributed from the apical to the luminal side of epithelial cells and co-localized with lymphatic lacteals within the intestinal villi (see FIG. 1H).

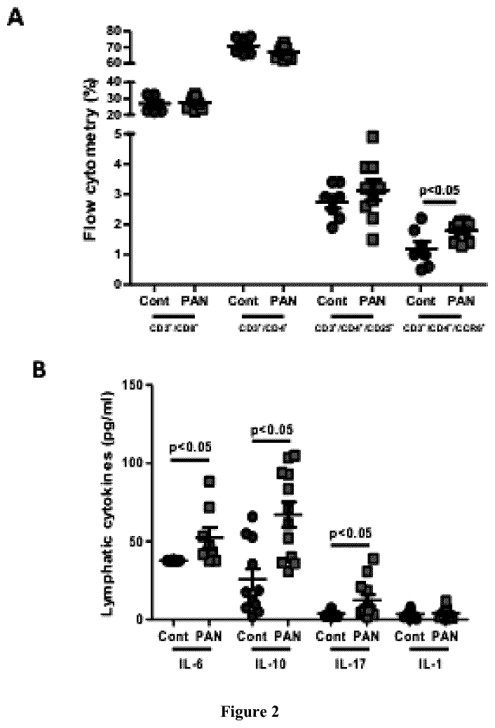

PAN injury affected the immune cell composition of mesenteric lymph, increasing the number of Th17 cells (CD3⁺CD4⁺CCR6⁺) (see FIG. 2A). PAN lymph had significantly elevated cytokines, including IL-6, IL-10 and IL-17 (see FIG. 2B). Notably, plasma cytokines obtained at the same time and assayed together with lymph samples were not different between PAN vs controls. These results demonstrate that proteinuric kidney injury increases mesenteric lymph flow rate, lymph lipids, lipoproteins, immune cells, and cytokines, but that many of these changes are not paralleled by changes in plasma.

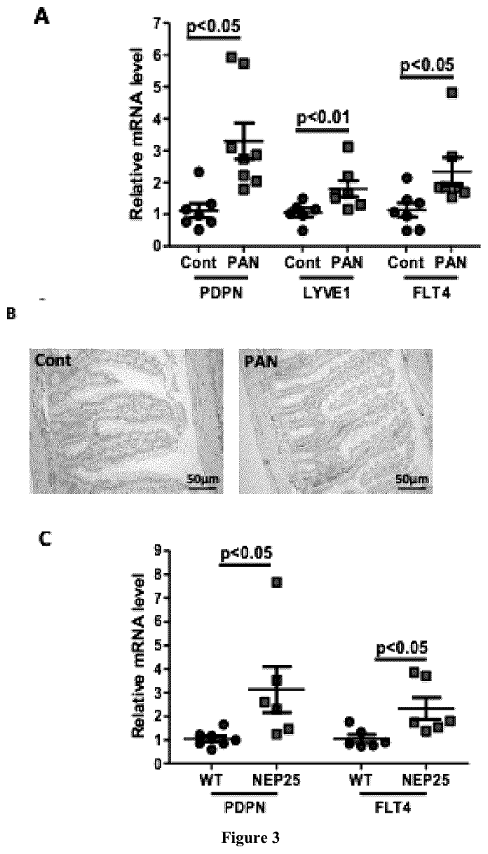

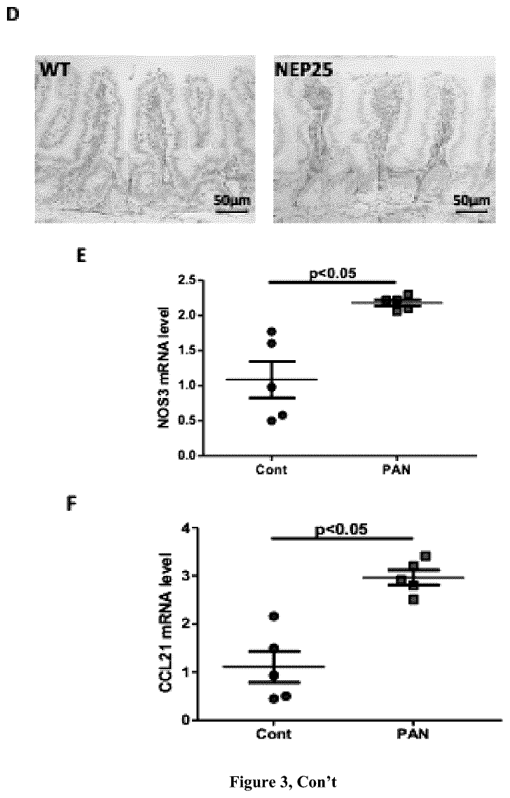

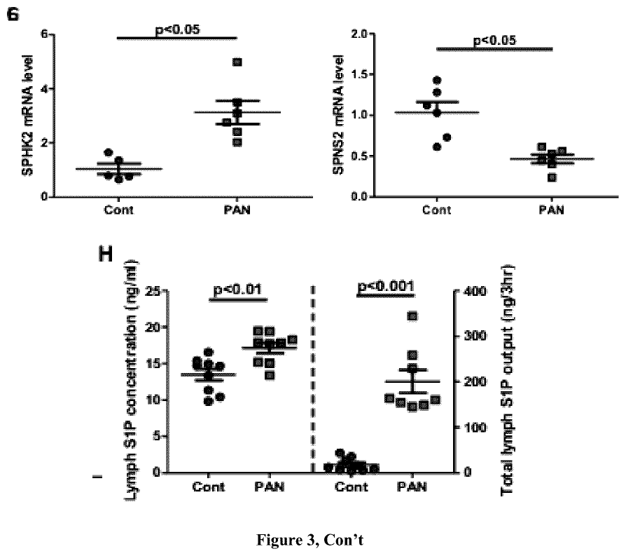

The present inventors also show that proteinuric kidney injury expands the intestinal lymphatic vascular network and alters the lymphatic endothelial cell phenotype. Lymphangiogenic markers, including podoplanin, LYVE-1 and VEGFR3, were all significantly increased in the ileum of PAN vs controls (see FIG. 3A). Complementing higher mRNAs, the ileum in PAN showed greater podoplanin-positive lymphatic vessels by IHC vs controls (see FIG. 3B). These findings were corroborated by results in transgenic NEP25 mice which showed increased ileal gene expression of podoplanin and VEGFR3 (FLT4) vs wild type mice (see FIG. 3C). Similar to PAN rats, the ileum of proteinuric mice had increased podoplanin expression compared to un-injured mice (see FIG. 3D).

To determine if renal injury affected the lymphatic endothelial cells (LECs), expression of key genes was quantified by PCR from LECs isolated from the ileum of PAN and control rats. Endothelial-specific nitric oxide (Nos3), a critical mediator of vasodilation, was significantly increased in intestinal LECs isolated from PAN vs control rats (see FIG. 3E). PAN also significantly unregulated intestinal expression of the chemokine CCL21, a key mediator of immune cell recruitment (see FIG. 3F). Podoplanin-positive LECs isolated from PAN ilea also showed higher SPHK2 and decreased SPNS2 expression—two key regulators of SP production, which in turn stimulates lymphocyte migration and survival (see FIG. 3G). The mesenteric lymph of PAN contained more SP vs controls (see FIG. 3H). Together, these results support the concept that proteinuric kidney injury increases key genes in intestinal LECs involved in lymphangiogenesis, vasodilation and immune cell chemoattraction.

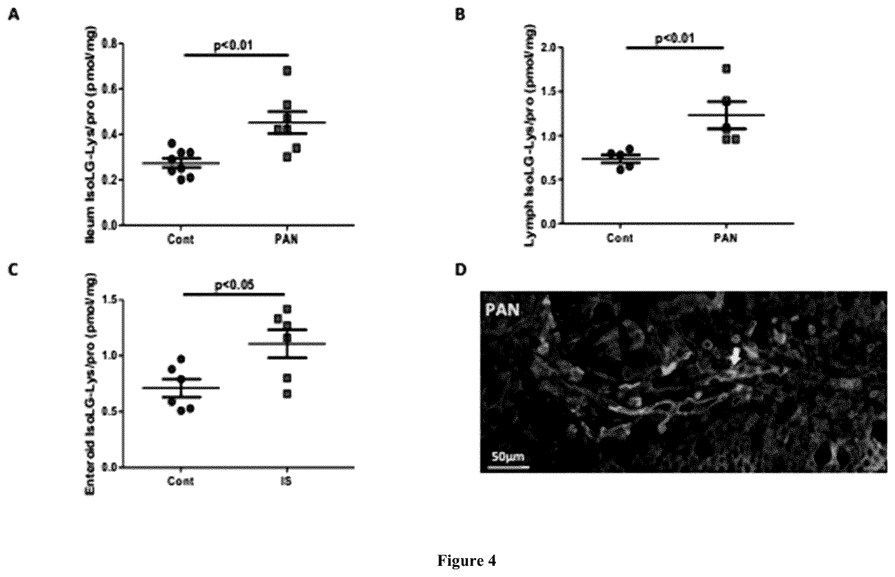

The present inventors also show that proteinuric kidney injury increases IsoLG-modified lipoproteins that regulate lymphatic endothelial cells and vessel dynamics. Kidney injury increases oxidative stress and lipid peroxidation that can generate a diverse family of lipid aldehydes, including IsoLG, which compromise apoAI functionality. PAN rats had more total IsoLG-lysine content in the ileum vs controls (see FIG. 4A). PAN rats also showed a significant increase in IsoLG-lysine levels in the mesenteric lymph (see FIG. 4B), but not in plasma (PAN:0.20±0.07 vs Cont:0.15±0.03 pmol/mg protein, pNS). Interestingly, IsoLG adduct production was significantly increased in cultured enteroids exposed to the uremic toxin, indoxyl sulfate (IS), compared to vehicle-treated enteroids (see FIG. 4C). Indeed, co-staining of the ileum for both apoAI and IsoLG showed overlap and co-localization of the epitopes in ilea of PAN rats (see FIG. 4D).

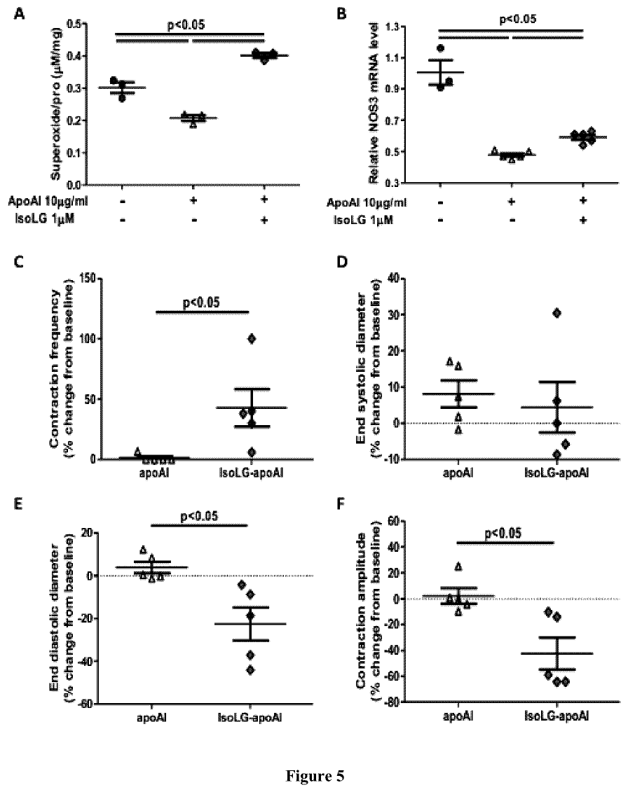

To determine if IsoLG-apoAI directly affects lymphatics, cultured LECs were exposed to IsoLG-apoAI. IsoLG-apoAI caused significantly greater production of ROS compared to unmodified apoAI (see FIG. 5A). Furthermore, LECs exposed to IsoLG-apoAI had increased Nos3 compared to LECs treated with unmodified apoAI (see FIG. 5B). In ex vivo studies, isolated mesenteric lymphatic vessels exposed to IsoLG-apoAI showed increased frequency of contractions compared to unmodified apoAI (see FIG. 5C). Although IsoLG-apoAI failed to significantly alter end-systolic diameter (ESD) (see FIG. 5D), end-diastolic diameter (EDD) was significantly decreased (see FIG. 5E) together with a significantly reduced amplitude of contraction in IsoLG-apoAI treated lymphatics (see FIG. 5F). These studies reveal a direct effect of IsoLG-apoAI on lymphatic vessel dynamics which are distinct from native apoAI. The results also suggest that frequency of contraction is a driving force in the increased lymph flow in vivo.

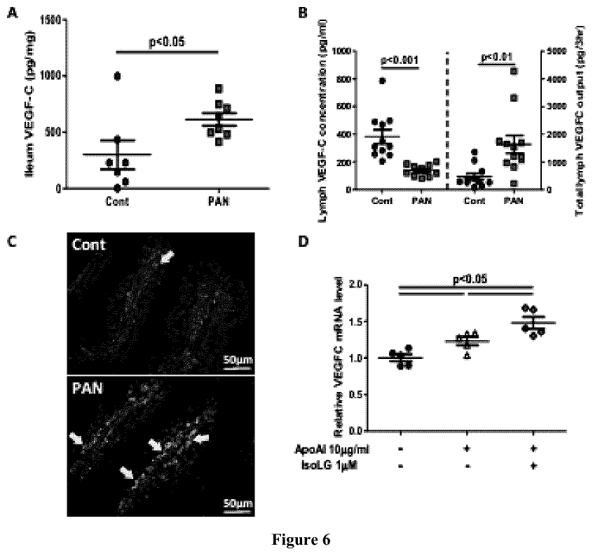

VEGF-C is the major growth factor promoting lymphangiogenesis. VEGF-C protein levels were significantly increased in the ileum of PAN vs control rats (see FIG. 6A). Importantly, VEGF-C protein levels were significantly increased in mesenteric lymph of PAN (see FIG. 6B), while no difference was observed in plasma VEGF-C in PAN vs control rats (PAN:11.03±0.76 pg/ml vs Cont:10.91±0.59 pg/ml, pNS). PAN had more CD68+cells (macrophages) in the intestinal lacteals that co-localized with VEGF-C protein (FIG. 6C). Increased VEGF-C protein content in lymph is likely linked to IsoLG-modification on lymphatic HDL, as macrophages treated with IsoLG-apoAI showed significantly increased VEGFC mRNA expression compared to unmodified apoAI (see FIG. 6D). These data support the hypothesis that the ileum is likely a source for increased VEGF-C levels in intestinal lymphatics and that intestinal macrophages likely contribute to the observed increase.

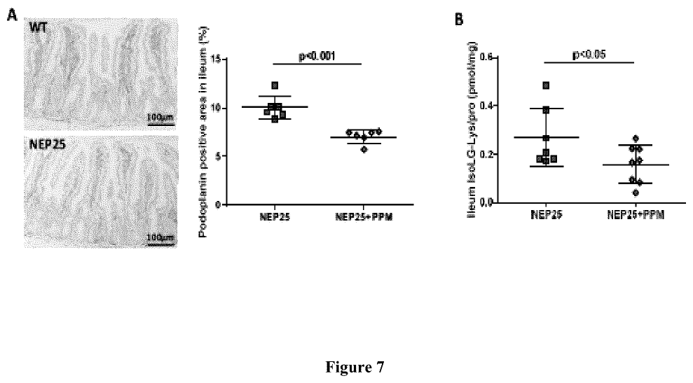

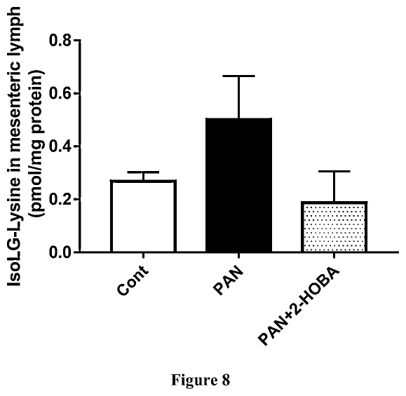

Compared with untreated NEP25 mice, NEP mice treated with compounds of the present invention showed significantly reduced intestinal podoplanin expression (FIG. 7A). A compound of the present invention also decreased the level of IsoLG adducts in the ileum (FIG. 7B). FIG. 8 shows another compound of the present invention reducing IsoLG in mesenteric lymph.

Experimental and clinical data have firmly established that kidney injury has detrimental consequences on distant organs such as heart, lungs and intestines. In the kidney-intestinal crosstalk, studies have primarily focused on the effects of kidney disease on the intestinal microbiome and barrier dysfunction. Aspects of the present invention show the effects of proteinuric kidney injury on intestinal lymphatics, which are central in absorption, metabolism, and transport of lipids and lipoproteins as well as in regulating immunity and inflammation. Using two models, the present inventors demonstrated that proteinuric kidney injury augments intestinal lymphangiogenesis, mesenteric lymph flow, lymphatic vessel contractions, and activation of lymphatic endothelial cells. The composition of the mesenteric lymph was also altered, with increased cytokines and immune cells. The present inventors demonstrated that kidney injury resulted in increased enteric production of IsoLG that can adduct local apoAI. The gut-originating IsoLG-apoAI then, directly and indirectly e.g., by stimulating VEGF-C, eNOS, ROS, alter growth and dynamics of the lymphatic vascular network and thus increase transmission of potentially harmful bioactive elements. Together, these data point to a new pathway in the crosstalk between kidneys and gut that underlies adverse systemic consequences of kidney disease with the intestinal lymphatic network as a conduit and IsoLG-apoAI as a novel mediator of these effects.

Two different proteinuric kidney injury models have significantly expanded intestinal lymphangiogenesis, evidenced by increased mRNA and immunostaining for lymphatic endothelial markers podoplanin, LYVE-1, and VEGFR3. Kidney injury also altered the phenotype of intestinal LECs. Podoplanin-positive LECs isolated from ilea of PAN rats had significantly increased Nos3 mRNA, results consistent with previous studies showing that LECs elaboration of eNOS is a major factor in lymphatic dilation. The data are in keeping with the striking increase in mesenteric lymph flow observed in PAN rats vs controls and fit observations that eNOS amplifies trafficking of lymphocytes and other immune cells, directing them to lymph nodes for antigen presentation and initiation of innate and adaptive immune responses. LECs can generate a chemokine gradient, especially CCL21 that recruits dendritic cells, macrophages and T-lymphocyte subsets into the lymphatic network. The present inventors showed that ileal podoplanin-positive LECs in PAN have increased CCL21 expression. Intestinal LECs showed increase in other factors regulating immune cell trafficking, e.g., SIP and S1PR1. Proteinuric kidney injury increased levels of potentially toxic immune cells (Th17 lymphocytes) and cytokines (IL-6, IL-10, IL-17). Together, our data demonstrate that proteinuric kidney injury expands the intestinal lymphatic network that then enhances lymph flow from the intestines. We also show that proteinuric kidney injury results in activated lymphatic vessel LECs with increased expression of vasodynamic mediators and chemoattractants of immune/inflammatory cells.

Cholesterol and triglycerides concentrations were lower in mesenteric lymph from PAN rats vs controls. However, in the face of augmented lymph flow, PAN rats had greater total mesenteric output of cholesterol and triglycerides, which may contribute to the severe mixed dyslipidemia characteristic of proteinuric diseases. Cholesterol and triglycerides become incorporated into chylomicrons or bind to apoAI in HDL. Because intestines synthesize a third of the total apoAI, we next examined the effects of PAN on intestinal apoAI. To limit the production of dietary lipoproteins, animals in our study were fasted. Our data failed to show differences in ileal apoAI protein concentration in lymph of PAN vs controls, however, IHC revealed apoAI redistributed from the apical to the luminal side in epithelial cells, co-localizing with lymphatic lacteals. It is possible that redistribution and/or enhanced secretion together with increased lymph flow, contributed to increased mesenteric output of apoAI. In addition to greater mesenteric output of apoAI, our study showed that in the early stages of kidney injury, mesenteric lymph contains larger HDL particles, more cytokines, higher VEGF-C, and added IsoLG (discussed below). Interestingly, increased levels of many of the molecules in PAN lymph were not seen in simultaneously obtained plasma of PAN vs control rats, data that support the idea that at least in the early stages following kidney injury, the intestines are a source of the potentially harmful molecules.

Proteinuric kidney injury caused intestinal generation of reactive peroxidation product IsoLG, which is a powerful modifier of apoAI/HDL that degrades many of its beneficial actions, including decreased ability to bind LPS, efflux cellular cholesterol, and inhibit cytokine response. IsoLG modifications of apoAI/HDL have been linked to the pathogenesis of sepsis, hypertension and CVD. ROS are increased in these conditions, and are powerful stimuli for IsoLG generation. The present inventors found that mesenteric lymph in PAN rats is enriched in IsoLG. Our data also reveal that indoxyl sulfate, a toxin originating in the gut known to stimulate ROS, significantly increased IsoLG-adducts in cultured ileal organoids. Since organoids encompass different cells within the ileum, the specific cell type responsible for producing IsoLG is uncertain. The data are in keeping with our in vivo results showing increased IsoLG content in ileal wall and mesenteric lymph of PAN rats vs controls. Indeed, we demonstrated that IsoLG-apoAI localized to ileal lymph vessels by double staining for lymphatic cell markers and IsoLG. Thus, the study indicates that proteinuric injury increases intestinal IsoLG production which can then modify local apoAI, although both intestinally-produced and circulation-derived apoAI particles could be modified by IsoLG.

The data indicate that intestinal lymphatics are not only a conduit for lipoprotein transport but present a target for their effects. Previous studies have described that apoAI/HDL regulate lymphangiogenesis and lymphatic integrity. The present inventors examined whether the effects of IsoLG-apoAI on lymphatic vessels or LECs differ from normal apoAI. IsoLG-apoAI increased Nos3 mRNA in cultured LECs compared to unmodified apoAI. The results complement the inventors' in vivo results that LECs isolated from PAN ilea have increased Nos3 mRNA. IsoLG-apoAI also changed functionality of isolated mesenteric lymphatic vessels, including blunted vasoactivity and greater contraction frequency vs unmodified apoAI. Although the ex vivo assessment of dynamics in individual lymphangions do not include contributions from innervation, circulating cytokines, or lymph flow, the approach reveals the direct impact of IsoLG-apoAI with minimal input from other variables. Together, the results show IsoLG-apoAI can stimulate LEC production of vasodilators and cause the lymphatic vessels to be more flaccid. Nonetheless, the concurrent increase in the contraction frequency may promote greater lymph flow and thus greater delivery of intestinally generated molecules and cells documented in PAN rats vs controls.

In addition to directly affecting LECs, IsoLG-apoAI also altered other cell types that modulate the lymphatic network. VEGF-C level was increased in the intestine and mesenteric lymph of PAN rats vs controls, and lymphangiogenesis and podoplanin immunostaining were increased in ilea of both PAN rats and NEP25 mice. While we did not specifically examine the source of VEGF-C in our proteinuric injury models, macrophages have long been recognized as an important source for VEGF-C. Macrophage depletion or blockade of VEGF-C signaling has been shown to diminish lymphangiogenesis. In the current study, macrophage infiltration of intestinal villi colocalized with VEGF-C in PAN. These data support the hypothesis that in proteinuric injury, macrophages are an important source for intestinal VEGF-C. Our results add the original observation that IsoLG-apoAI can increase macrophage expression of VEGFC. Together, these data suggest that mechanisms for IsoLG-apoAI modulation of lymphatic vessels involve direct modulation of LEC genes, as well as indirect increase of the lymphatic network through macrophage production of VEGF-C.

Thus, the present inventors discovered a novel connection between kidney disease and intestinal response (FIG. 7). Aspects of the invention show that kidney injury stimulates enteric production of IsoLG adducts which modify intestinally originating apoAI/HDL. Other aspects of the invention show that the intestinal/mesenteric lymphatic network serves as both target and perpetrator of IsoLG-HDL's effects by augmenting lymphangiogenesis, lymphatic vessel contractions, LEC activation, and increased lymph flow. The net effect is greater delivery of intestinally derived molecules which constitute a new mechanism for adverse kidney-intestine crosstalk.

### METHODS

### Animals

Adult male Sprague Dawley rats (200-225 g, Charles River) were housed under a 12-h light/dark cycle with free access to normal rat chow and water. Kidney injury was induced by a single injection of puromycin aminoglycoside (PAN) (125 mg/kg body weight, i.p.) while saline-injected rats served as controls (Cont). Eight days after injection, rats were sacrificed and blood, urine, and tissues were harvested. We also studied adult male (12 weeks old) Nphs1-hCD25 transgenic (NEP25, C57 bl/6 background) mice expressing human CD25 on podocytes can be selectively injured by injection of recombinant immunotoxin, anti-Tac (Fv)-PE38 (LMB2, 1 ng/g BW, i.v. generously provided by Dr. Ira Pastan) that results in proteinuria. These and wild type control mice were housed under normal conditions with free access to regular rodent chow and water. Two weeks after LMB2, mice were sacrificed and blood, urine and tissues harvested. All animal procedures were approved by the Institutional Animal Care and Use Committee at Vanderbilt University.

### Assessments of Lymph, Plasma, and Urine Composition

Mesenteric lymph was collected in a subset of conscious rats following cannulation of the mesenteric lymph duct. The rats were placed in Bollman cages in a temperature- and humidity-controlled incubator and lymph collected hourly for at least 3 h.

Albumin (Exocell), apoAI (Mybiosource), sphingosine-1 phosphate (S1P) (Mybiosource) and VEGF-C levels (Mybiosource) were measured by ELISA. Albuminuria was measured as urine albumin-to-creatinine ratio (ACR) (Nephrat II, Exocell) and QuantiChrom™ Creatinine Assay Kit (Bioassay Systems), respectively. Plasma and lymph total cholesterol and triglycerides were measured enzymatically (Cliniqa). HDL and LDL fractions were isolated from plasma and lymph by density-gradient ultracentrifugation after adjustment with potassium bromide. Lipoprotein particle size was evaluated by NMR methodology (Liposcience). Colorimetric assays were used to measure protein (BCA, ThermoFisher). Lipoproteins in plasma and lymph were separated by size-exclusion chromatography (SEC) using an Akta Pure fast-protein liquid chromatography (FPLC) system (GE Healthcare). Total IsoLG-protein adduct by LC/MS was measured as IsoLG-lysine after complete proteolytic digestion of lymph samples.

Plasma and lymph levels of interleukin-6 (IL-6), IL-10, IL-17, and IL-1 were determined by Luminex multiplex. Immune cells in lymph were quantitated by flow cytometry. The samples were incubated with Fc blocking antibody (BD Biosciences), then incubated with BV421-conjugated anti-CD3 (BD Biosciences), PE/Cy7-conjugated anti-CD4 (Biolegend), Percp-conjugated anti-CD8 (Biolegend), Alexa Flour 488-conjugated anti-CD25 (Biolegend) or PE-conjugated anti-CCR6 (R&D Systems)×30 min at room temperature. Cells were analyzed on a FACSCanto II cytometer with FACSDiva software (BD Biosciences).

### Immunostaining on Intestinal Tissue

Ileal sections were fixed in 4% paraformaldehyde/PBS, dehydrated, paraffin embedded, and cut for immunostaining. We focused on the small intestine because lymph drains into the mesenteric lymph vessels and because the ileum is critical in apoAI synthesis. For podoplanin staining, ileal sections were incubated with mouse anti-podoplanin antibody (1:1000, Novus) overnight followed by HRP anti-mouse antibody (Vector Laboratories) and the signal visualized by diaminobenzidine. Mouse ileal sections were incubated with hamster anti-podoplanin antibody (1:2000, ThermoFisher) overnight followed by biotinylated anti-hamster antibody (Vector Laboratories) and ABC reagent, and the signal visualized by diaminobenzidine.

Double staining for apoAI and podoplanin used citrate buffer for antigen retrieval followed by primary antibody apoAI (1:200; Novus) overnight. ImmPRESS reagent (Vector Laboratories) and Alexa Fluor 546 Tyamide SuperBoost (Invitrogen) were used as secondary antibodies. Sections were incubated with mouse anti-rat podoplanin overnight followed by anti-mouse horseradish peroxidase (HRP) (ImmPRESS) and Alexa Fluor 488 Tyamide SuperBoost. Double staining for IsoLG and apoAI used citrate buffer for antigen retrieval, followed by anti-IsoLG (1:10; a generous gift from Dr. Annet Kirabo) overnight. The second antibody used ImmPACT® Vector® Red Alkaline Phosphatase Substrate kit (Vector Laboratories) as a chromogen. Sections were then incubated with rabbit anti-rat apoAI overnight, followed by anti-rabbit horseradish peroxidase (HRP) (ImmPRESS) and Alexa Fluor 488 Tyamide SuperBoost. Double-staining for CD68 and VEGF-C used citrate buffer for antigen retrieval, followed by biotinylated primary antibody targeting CD68 (1:10; BioRad) overnight. The second antibodies were ABC reagent and Alexa Fluor 488 Tyamide SuperBoost. Sections were then incubated with mouse anti-rat VEGF-C (1:200; Abcam) overnight, followed by anti-mouse horseradish peroxidase (HRP) (ImmPRESS) and Alexa Fluor 546 Tyamide SuperBoost.

### Measurement of Lymphatic Dynamics Ex Vivo

Mesenteric lymphatic vessels were collected and mounted in a perfusion chamber. Chambers were placed on inverted microscopes equipped with a digital image capture system (IonOptix) to record pre-valve intraluminal diameters and frequency of contractions. Vessels were warmed to 37° C., pressurized to 0.5 mmHg using a column of Krebs buffer, and allowed to equilibrate (20-60 minutes). Viable vessels were then pressurized in a stepwise manner to a constant pressure of 3.5 mmHg, then exposed to purified apoAI or modified apoAI using 1 molar equivalent synthetic IsoLG or vehicle (DMSO). Fresh Krebs buffer was circulated to facilitate wash out. Next vessels were exposed to IsoLG-modified apoAI. After each challenge, lumen diameters were allowed to plateau (40-60 minutes).

### Characterization of Intestinal Lymphatic Endothelial Cells

Ilea from PAN and control rats were minced, then incubated with collagenase type D (Roche Applied Science), 1 mL HBSS medium and 10 ml/mL DNase for 1 h. The tissue was filtered using 70 μm then 40 μm sieves. Cells were resuspended and incubated with podoplanin-selective reagent (Novus). Lymphatic endothelial cells, i.e., podoplanin-positive cells, were isolated using the EasyEights magnetic cell separation system (Stemcell Technologies).

Total RNA was isolated from ileum lysates and ileal podoplanin-positive cells by RNase Mini kit (QIAGEN). Reverse transcription was performed using High-Capacity cDNA Reverse Transcription Kit (Applied Biosystems). Quantitative real-time PCR was performed in a total reaction volume of 25 μL using 12.5 μL Universal Master Mix II, 1.25 μL forward and reverse primers [podoplanin (PDPN), lymphatic endothelial receptor (LYVE1), vascular endothelial growth factor receptor 3 (VEGFR3, FLT4), sphingosine kinase 2 (SPHK2), sphingolipid transporter 2 (SPNS2), C-C motif chemokine ligand 21 (CCL21) and nitric oxide synthase 3 (eNOS, Nos3)] (ThermoFisher) and 11.25 μL cDNA (10 ng/μL). Quantitative real-time PCR used the CFX96™ Real-Time PCR Detection System (RT-PCR, Bio-Rad) with the following cycling parameters: polymerase activation for 10 min at 95° C. and amplification for 40 cycles of 15 s at 95° C. and 60 s at 60° C. Experimental cycle threshold (Ct) values were normalized to 18S measured on the same plate, and fold differences in gene expression were determined by the 2^{-ΔΔCt} method

### Organoid and Cell Culture

Intact perfused intestine was used to generate ileal organoids. Cultured ileal organoids were incubated in medium with or without indoxyl sulfate (1 mmol/L, Sigma) for 3 days. Total protein was extracted for quantitation of IsoLG.

Primary adult dermal lymphatic endothelial cells (LECs) (HMVEC-DLyAd, Lonza) were cultured with conditioned growth medium (Lonza). Cells at passage 5-6 with ˜70% confluence were starved in serum-free medium overnight, then incubated with unmodified or IsoLG-modified apoAI (apoAI: 10 μg/ml, IsoLG: 1 μM/L) for 18 h. Previously, we showed that this concentration of IsoLG yields levels of IsoLG-lysine adducts observed in vivo and does not produce unreacted IsoLG. Quantification of eNOS (Nos3) and β-actin (ACTB) mRNA was performed by RT-PCR and production of superoxide was assessed by high-performance liquid chromatography.

THP-1 cells were plated and differentiated into macrophages by RPMI 1670 containing 10% FBS and 50 ng/ml phorbol 12-myristate 13-acetate for 3 days. Cells were incubated with unmodified or IsoLG-modified apoAI (apoAI: 10 μg/ml, IsoLG: 1 μM/L) for 48 h. VEGFC mRNA was assessed by RT-PCR.

### Statistical Analysis

Data are expressed as mean±SEM. Differences were determined by unpaired t-test, and p<0.05 was deemed significant.

All publications mentioned herein, including those listed below, are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

### REFERENCES

1. Vaziri N D, Zhao Y Y, Pahl M V. Altered intestinal microbial flora and impaired epithelial barrier structure and function in CKD: the nature, mechanisms, consequences and potential treatment. Nephrology, dialysis, transplantation : official publication of the European Dialysis and Transplant Association—European Renal Association 2016; 31: 737-746.
2. Evenepoel P, Poesen R, Meijers B. The gut-kidney axis. Pediatric nephrology 2017; 32: 2005-2014.
3. Andersen K, Kesper M S, Marschner J A, et al. Intestinal Dysbiosis, Barrier Dysfunction, and Bacterial Translocation Account for CKD-Related Systemic Inflammation Journal of the American Society of Nephrology : JASN 2017; 28: 76-83.
4. Wikoff W R, Nagle M A, Kouznetsova V L, et al. Untargeted metabolomics identifies enterobiome metabolites and putative uremic toxins as substrates of organic anion transporter 1 (Oat1). Journal of proteome research 2011; 10: 2842-2851.
5. de Loor H, Bammens B, Evenepoel P, et al. Gas chromatographic-mass spectrometric analysis for measurement of p-cresol and its conjugated metabolites in uremic and normal serum. Clinical chemistry 2005; 51: 1535-1538.
6. McIntyre C W, Harrison L E, Eldehni M T, et al. Circulating endotoxemia: a novel factor in systemic inflammation and cardiovascular disease in chronic kidney disease. Clinical journal of the American Society of Nephrology : CJASN 2011; 6: 133-141.
7. Randolph G J, Miller N E. Lymphatic transport of high-density lipoproteins and chylomicrons. The Journal of clinical investigation 2014; 124: 929-935.
8. Ramezani A, Massy Z A, Meijers B, et al. Role of the Gut Microbiome in Uremia: A Potential Therapeutic Target. American journal of kidney diseases : the official journal of the National Kidney Foundation 2016; 67: 483-498.
9. Castillo-Rodriguez E, Fernandez-Prado R, Esteras R, et al. Impact of Altered Intestinal Microbiota on Chronic Kidney Disease Progression. Toxins 2018; 10.
10. Nurmi H, Saharinen P, Zarkada G, et al. VEGF-C is required for intestinal lymphatic vessel maintenance and lipid absorption. EMBO molecular medicine 2015; 7: 1418-1425.
11. Becker F, Kurmaeva E, Gavins F N, et al. A Critical Role for Monocytes/Macrophages During Intestinal Inflammation-associated Lymphangiogenesis. Inflammatory bowel diseases 2016; 22: 1326-1345.
12. Vaziri N D, Deng G, Liang K. Hepatic HDL receptor, SR-B 1 and Apo A-I expression in chronic renal failure. Nephrology, dialysis, transplantation : official publication of the European Dialysis and Transplant Association—European Renal Association 1999; 14: 1462-1466.
13. Fu J, Bonder M J, Cenit M C, et al. The Gut Microbiome Contributes to a Substantial Proportion of the Variation in Blood Lipids. Circulation research 2015; 117: 817-824.
14. Mistry R H, Verkade H J, Tietge U J. Reverse Cholesterol Transport Is Increased in Germ-Free Mice-Brief Report. Arteriosclerosis, thrombosis, and vascular biology 2017; 37: 419-422.
15. Davies S S, May-Zhang L S. Isolevuglandins and cardiovascular disease. Prostaglandins & other lipid mediators 2018; 139: 29-35.
16. May-Zhang L S, Yermalitsky V, Huang J, et al. Modification by isolevuglandins, highly reactive gamma-ketoaldehydes, deleteriously alters high-density lipoprotein structure and function. The Journal of biological chemistry 2018; 293: 9176-9187.
17. Yamamoto S, Zhong J, Yancey P G, et al. Atherosclerosis following renal injury is ameliorated by pioglitazone and losartan via macrophage phenotype. Atherosclerosis 2015; 242: 56-64.
18. Tsuchida Y, Zhong J, Otsuka T, et al. Lipoprotein modulation of proteinuric renal injury. Laboratory investigation; a journal of technical methods and pathology 2019; 99: 1107-1116.
19. Florens N, Calzada C, Lyasko E, et al. Modified Lipids and Lipoproteins in Chronic Kidney Disease: A New Class of Uremic Toxins. Toxins 2016; 8.
20. Ikizler T A, Morrow J D, Roberts L J, et al. Plasma F2-isoprostane levels are elevated in chronic hemodialysis patients. Clinical nephrology 2002; 58: 190-197.
21. Kronenberg F. HDL in CKD-The Devil Is in the Detail. Journal of the American Society of Nephrology : JASN 2018; 29: 1356-1371.
22. Bisoendial R, Tabet F, Tak P P, et al. Apolipoprotein A-I Limits the Negative Effect of Tumor Necrosis Factor on Lymphangiogenesis. Arteriosclerosis, thrombosis, and vascular biology 2015; 35: 2443-2450.
23. Milasan A, Jean G, Dallaire F, et al. Apolipoprotein A-I Modulates Atherosclerosis Through Lymphatic Vessel-Dependent Mechanisms in Mice. Journal of the American Heart Association 2017; 6.
24. Grond J, Weening J J, van Goor H, et al. Application of puromycin aminonucleoside and adriamycin to induce chronic renal failure in the rat. Contributions to nephrology 1988; 60: 83-93.
25. Arciello A, Piccoli R, Monti D M. Apolipoprotein A-I: the dual face of a protein. FEBS letters 2016; 590: 4171-4179.
26. Stepanovska B, Huwiler A. Targeting the S113 receptor signaling pathways as a promising approach for treatment of autoimmune and inflammatory diseases. Pharmacological research 2019: 104170.
27. Himmelfarb J, Stenvinkel P, Ikizler TA, et al. The elephant in uremia: oxidant stress as a unifying concept of cardiovascular disease in uremia. Kidney international 2002; 62: 1524-1538.
28. Husain-Syed F, McCullough P A, Birk H W, et al. Cardio-Pulmonary-Renal Interactions: A Multidisciplinary Approach. Journal of the American College of Cardiology 2015; 65: 2433-2448.
29. Lane K, Dixon J J, MacPhee I A, et al. Renohepatic crosstalk: does acute kidney injury cause liver dysfunction? Nephrology, dialysis, transplantation : official publication of the European Dialysis and Transplant Association—European Renal Association 2013; 28: 1634-1647.
30. Hoste E A J, Kellum J A, Selby N M, et al. Global epidemiology and outcomes of acute kidney injury. Nature reviews Nephrology 2018; 14: 607-625.
31. Turner J R. Intestinal mucosal barrier function in health and disease. Nature reviews Immunology 2009; 9: 799-809.
32. Jang J Y, Koh Y J, Lee S H, et al. Conditional ablation of LYVE-1+cells unveils defensive roles of lymphatic vessels in intestine and lymph nodes. Blood 2013; 122: 2151-2161.
33. Datar S A, Gong W, He Y, et al. Disrupted NOS signaling in lymphatic endothelial cells exposed to chronically increased pulmonary lymph flow. American journal of physiology Heart and circulatory physiology 2016; 311: H137-145.
34. Karnezis T, Shayan R, Caesar C, et al. VEGF-D promotes tumor metastasis by regulating prostaglandins produced by the collecting lymphatic endothelium. Cancer cell 2012; 21: 181-195.
35. Weber M, Hauschild R, Schwarz J, et al. Interstitial dendritic cell guidance by haptotactic chemokine gradients. Science 2013; 339: 328-332.
36. Card C M, Yu S S, Swartz M A. Emerging roles of lymphatic endothelium in regulating adaptive immunity. The Journal of clinical investigation 2014; 124: 943-952.
37. Brunham L R, Kruit J K, Iqbal J, et al. Intestinal ABCA1 directly contributes to HDL biogenesis in vivo. The Journal of clinical investigation 2006; 116: 1052-1062.
38. Iyer R S, Ghosh S, Salomon R G. Levuglandin E2 crosslinks proteins. Prostaglandins 1989; 37: 471-480.
39. Murthi K K, Friedman L R, Oleinick N L, et al. Formation of DNA-protein cross-links in mammalian cells by levuglandin E2. Biochemistry 1993; 32: 4090-4097.
40. Poliakov E, Brennan M L, Macpherson J, et al. Isolevuglandins, a novel class of isoprostenoid derivatives, function as integrated sensors of oxidant stress and are generated by myeloperoxidase in vivo. FASEB journal : official publication of the Federation of American Societies for Experimental Biology 2003; 17: 2209-2220.
41. Wu J, Saleh M A, Kirabo A, et al. Immune activation caused by vascular oxidation promotes fibrosis and hypertension. The Journal of clinical investigation 2016; 126: 1607.
42. Salomon R G, Kaur K, Batyreva E. Isolevuglandin-protein adducts in oxidized low density lipoprotein and human plasma: a strong connection with cardiovascular disease. Trends in cardiovascular medicine 2000; 10: 53-59.
43. Zhang Y, Zanotti I, Reilly M P, et al. Overexpression of apolipoprotein A-I promotes reverse transport of cholesterol from macrophages to feces in vivo. Circulation 2003; 108: 661-663.
44. Cursiefen C, Chen L, Borges L P, et al. VEGF-A stimulates lymphangiogenesis and hemangiogenesis in inflammatory neovascularization via macrophage recruitment. The Journal of clinical investigation 2004; 113: 1040-1050.
45. Kim H, Kataru R P, Koh G Y. Inflammation-associated lymphangiogenesis: a double-edged sword? The Journal of clinical investigation 2014; 124: 936-942.
46. Matsusaka T, Xin J, Niwa S, et al. Genetic engineering of glomerular sclerosis in the mouse via control of onset and severity of podocyte-specific injury. Journal of the American Society of Nephrology : JASN 2005; 16: 1013-1023.
47. Matsusaka T, Sandgren E, Shintani A, et al. Podocyte injury damages other podocytes. Journal of the American Society of Nephrology: JASN 2011; 22: 1275-1285.
48. Albaugh V L, Banan B, Antoun J, et al. Role of Bile Acids and GLP-1 in Mediating the Metabolic Improvements of Bariatric Surgery. Gastroenterology 2019; 156: 1041-1051 e1044.
49. Jerome W G, Cox B E, Griffin E E, et al. Lysosomal cholesterol accumulation inhibits subsequent hydrolysis of lipoprotein cholesteryl ester. Microscopy and microanalysis : the official journal of Microscopy Society of America, Microbeam Analysis Society, Microscopical Society of Canada 2008; 14: 138-149.
50. Yermalitsky V N, Matafonova E, Tallman K, et al. Simplified LC/MS assay for the measurement of isolevuglandin protein adducts in plasma and tissue samples. Analytical biochemistry 2019; 566: 89-101.
51. Green P H, Tall A R, Glickman R M. Rat intestine secretes discoid high density lipoprotein. The Journal of clinical investigation 1978; 61: 528-534.
52. Scallan J P, Hill M A, Davis M J. Lymphatic vascular integrity is disrupted in type 2 diabetes due to impaired nitric oxide signalling. Cardiovascular research 2015; 107: 89-97.
53. Zawieja S D, Castorena-Gonzalez J A, Dixon B, et al. Experimental Models Used to Assess Lymphatic Contractile Function. Lymphatic research and biology 2017; 15: 331-342.
54. Yao L, Wright M F, Farmer B C, et al. Fibroblast-specific plasminogen activator inhibitor-1 depletion ameliorates renal interstitial fibrosis after unilateral ureteral obstruction. Nephrology, dialysis, transplantation : official publication of the European Dialysis and Transplant Association—European Renal Association 2019.
55. Yang H C, Ma L J, Ma J, et al. Peroxisome proliferator-activated receptor-gamma agonist is protective in podocyte injury-associated sclerosis. Kidney international 2006; 69: 1756-1764.
56. Goldspink D A, Lu V B, Billing L J, et al. Mechanistic insights into the detection of free fatty and bile acids by ileal glucagon-like peptide-1 secreting cells. Molecular metabolism 2018; 7: 90-101.
57. Dikalova A, Clempus R, Lassegue B, et al. Noxl overexpression potentiates angiotensin II-induced hypertension and vascular smooth muscle hypertrophy in transgenic mice. Circulation 2005; 112: 2668-2676.
58. Yamamoto S, Yancey P G, Ikizler TA, et al. Dysfunctional high-density lipoprotein in patients on chronic hemodialysis. Journal of the American College of Cardiology 2012; 60: 2372-2379.

The invention thus being described, it would be obvious that the same can be varied in many ways. Such variations that would be obvious to one of ordinary skill in the art is to be considered as being bard of this disclosure.

Unless otherwise indicated, all numbers expressing quantities of ingredients, properties such as reaction conditions, and so forth used in the Specification are to be understood as being modified in all instances by the term “about.” Accordingly, unless indicated by the contrary, the numerical parameters set forth in the Specification and Claims are approximations that may vary depending upon the desired properties sought to be determined by the present invention.

Notwithstanding that the numerical ranges and parameters setting forth the broad scope of the invention are approximations, the numerical values set forth in the experimental sections or the example sections are reported as precisely as possible. Any numerical value, however, inherently contain certain errors necessarily resulting from the standard deviation found in their respective testing measurements.

We claim:

1. A method of treating proteinuric kidney injury, comprising:
identifying a subject in need of treatment of kidney damage; and
administering to said subject an effective isoLG scavenging amount of at least one compound of the following formula: 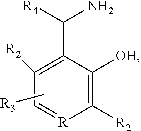
wherein:
R is N or C—R₂;
R₂ is independently selected from H, substituted or unsubstituted C₁-C₁₀ alkyl, C₁-C₁₀ alkoxy, hydroxymethyl, hydroxy;
R₃ is H, halogen, C₁-C₁₀ alkyl, alkoxy, hydroxyl, nitro;
R₄ is H, substituted or unsubstituted C₁-C₁₀ alkyl, carboxyl; and pharmaceutically acceptable salts thereof.

2. The method of claim 1, wherein the compound is 2-hydroxybenzylamine, methyl-2-hydroxybenzylamine, ethyl-2-hydroxybenzylamine, or 5′-O-pentyl-pyridoxamine.

3. The method of claim 1, wherein the compound is of the following formula: 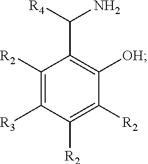
wherein:
R₂ is independently chosen from H, substituted or unsubstituted alkyl;
R₃ is H, halogen, alkyl, alkoxy, hydroxyl, nitro;
R₄ is H, substituted or unsubstituted alkyl, carboxyl; and pharmaceutically acceptable salts thereof.

4. The method of claim 1, wherein the proteinuric kidney injury is damage from kidney disease.

5. The method of claim 4, wherein the damage is to the intestinal lymphatic network.

6. The method of claim 4, wherein the damage is increased contractions of lymphatic vessels and activated lymphatic endothelial cells.

7. The method of claim 4, wherein the damage is disruptions in lymph transport and lymphatic vessel integrity.

8. The method of one of claims 1, wherein the compound is: 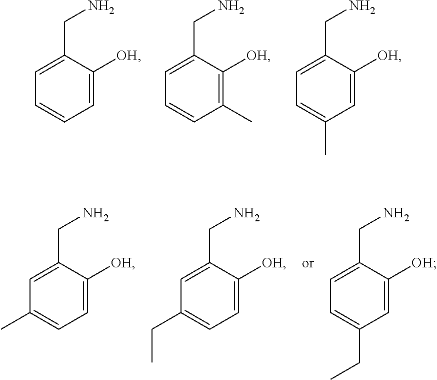
or a pharmaceutically acceptable salt thereof.

9. A method of modulating intestinal lymphatic function to ameliorate kidney injury or disease, comprising administering an effective isoLG scavenging amount of at least one compound of the following formula: 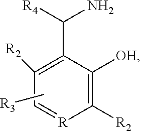
wherein:
R is N or C—R₂;
R₂ is independently selected from H, substituted or unsubstituted C₁-C₁₀ alkyl, C₁-C₁₀ alkoxy, hydroxymethyl, hydroxy;
R₃ is H, halogen, C₁-C₁₀ alkyl, alkoxy, hydroxyl, nitro;
R₄ is H, substituted or unsubstituted C₁-C₁₀ alkyl, carboxyl; and pharmaceutically acceptable salts thereof.

10. The method of claim 9, wherein the compound is 2-hydroxybenzylamine, methyl-2-hydroxybenzylamine, ethyl-2-hydroxybenzylamine, or 5′-O-pentyl-pyridoxamine.

11. The method of claim 9, wherein the compound is of the following formula: 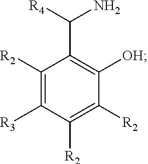
wherein:
R₂ is independently chosen from H, substituted or unsubstituted alkyl;
R₃ is H, halogen, alkyl, alkoxy, hydroxyl, nitro;
R₄ is H, substituted or unsubstituted alkyl, carboxyl; and pharmaceutically acceptable salts thereof.

12. A method of ameliorating systemic complications of kidney injury or disease, comprising administering an effective isoLG scavenging amount of at least one compound of the following formula: 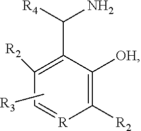
wherein:
R is N or C—R₂;
R₂ is independently selected from H, substituted or unsubstituted C₁-C₁₀ alkyl, C₁-C₁₀ alkoxy, hydroxymethyl, hydroxy;
R₃ is H, halogen, C₁-C₁₀ alkyl, alkoxy, hydroxyl, nitro;
R₄ is H, substituted or unsubstituted C₁-C₁₀ alkyl, carboxyl; and pharmaceutically acceptable salts thereof.

13. The method of claim 12, wherein the compound is 2-hydroxybenzylamine, methyl-2-hydroxybenzylamine, ethyl-2-hydroxybenzylamine, or 5′-O-pentyl-pyridoxamine.

14. The method of claim 12, wherein the compound is of the following formula: 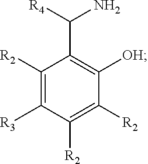
wherein:
R₂ is independently chosen from H, substituted or unsubstituted alkyl;
R₃ is H, halogen, alkyl, alkoxy, hydroxyl, nitro;
R₄ is H, substituted or unsubstituted alkyl, carboxyl; and pharmaceutically acceptable salts thereof.

15. The method of claim 12, wherein the IsoLG is in the intestinal lymphatic network.

16. The method of claim 12, wherein the systemic complication is cardiovascular, circulatory, or obesity-related.

17. A method of ameliorating intestinal lymphatic dysfunction, comprising administering an effective isoLG scavenging amount of at least one compound of the following formula: 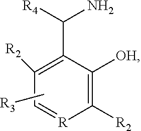
wherein:
R is N or C—R₂;
R₂ is independently selected from H, substituted or unsubstituted C₁-C₁₀ alkyl, C₁-C₁₀ alkoxy, hydroxymethyl, hydroxy;
R₃ is H, halogen, C₁-C₁₀ alkyl, alkoxy, hydroxyl, nitro;
R₄ is H, substituted or unsubstituted C₁-C₁₀ alkyl, carboxyl; and pharmaceutically acceptable salts thereof.

18. The method of claim 17, wherein the compound is 2-hydroxybenzylamine, methyl-2-hydroxybenzylamine, ethyl-2-hydroxybenzylamine, or 5′-O-pentyl-pyridoxamine.

19. The method of claim 17, wherein the compound is of the following formula: 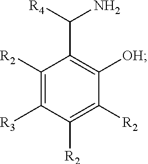
wherein:
R₂ is independently chosen from H, substituted or unsubstituted alkyl;
R₃ is H, halogen, alkyl, alkoxy, hydroxyl, nitro;
R₄ is H, substituted or unsubstituted alkyl, carboxyl; and pharmaceutically acceptable salts thereof.

20. The method of claim 17, wherein the dysfunction is intestinal lymphangiogenesis.
